(19) United States
(12) Patent Application Publication
(10) Pub. No.: US 2021/0386909 A1
(43) Pub. Date: Dec. 16, 2021

(54) MAMMAL-DERIVED MATRIX AS A SAFE BONE REPLACEMENT

(71) Applicant: IVORY GRAFT LTD., Holon (IL)

(72) Inventors: Larion SAPOZNIKOV, Ramat Gan (IL); Meir SAHAR, Holon (IL); Ruben TSATURYAN, Moscow (RU)

(21) Appl. No.: 17/285,940

(22) PCT Filed: Oct. 17, 2019
(86) PCT No.: PCT/IL2019/051125

§ 371 Date: Apr. 16, 2021

(30) Foreign Application Priority Data
Oct. 18, 2018 (IL) 262473

(51) Int. Cl. A61L 27/36

(52) U.S. Cl. CPC A61L 27/3608; A61L 27/3687; A61L 2430/02; A61L 27/365; A61L 27/3691

(57) ABSTRACT

The invention provides acellular matrix from mammalian teeth for preparing a safe graft or bone replacement in regenerative medicine.

### FIELD OF THE INVENTION

The present invention relates to a method of manufacturing a matrix for using as bone graft or bone replacement. Particularly, mammalian teeth or mammalian bone is processed to provide a safe xenograft in regenerative medicine.

### BACKGROUND OF THE INVENTION

Bone grafting is a surgical procedure that replaces missing bone and is widely used in regenerative medicine, for example in dental surgery, in treating fractures, or after resection of bone tumors (see, for example, The Merck Manual, 19th Ed., 2011, p. 364). Bone can regenerate but needs a graft as a scaffold. The graft may originate from the patient's own body—autograft—or it can come from other donor or from cadaveric bone—allograft; the graft may be synthetic. The graft material should preferably be osteoconductive, i.e. it should support the growth of the new bone on its surface; the material should preferably be also osteoinductive, i.e. it should induce the process of osteogenesis. Since tooth dentine is very similar to bone, extracted teeth were considered for preparing a bone graft; for example EP 2 462 899 relates to an alveolar bone graft prepared from patient's own tooth, processes by dewatering in alcohol, degreasing in ether, and decalcification in hydrochloric acid. It would be highly desirable to use a teeth scaffold material for any subject in need and not only for the tooth donor as an autograft, but a danger of strong immune responses or viral and other infections restricts the use of such grafts in other subjects of the same species (allograft), and still more the use of such grafts in subjects of a different species (xenograft). U.S. Pat. No. 9,610,383 attempts to overcome the problem by irradiating extracted teeth or bones by ultrasonic waves at a reduced pressure, followed by chemical treatments, including a treatment with hydrochloric acid. Still stricter safety regulations necessitate the development of new matrices for other than autograft applications. It is therefore an object of this invention to provide a matrix as a bone substitute for bone replacement applications.

It is another object of this invention to provide a method for producing a scaffold matrix for bone grafting, based on mammalian teeth and applicable instead of allograft.

It is still another object of this invention to provide a method for producing a scaffold matrix for bone grafting, based on mammalian teeth and applicable as xenograft.

It is a further object of this invention to provide a matrix based on mammalian teeth for safe use as a bone substitute in both allograft and xenograft applications.

Other objects and advantages of present invention will appear as description proceeds.

### SUMMARY OF THE INVENTION

The present invention provides a bone graft or bone replacement comprising acellular powder of crushed mammalian teeth or bone or parts thereof, extracted with an alcoholic hydroxide and heated to a temperature between 110 and 190° C. In one embodiment, the invention provides a bone graft or bone replacement comprising acellular powder of crushed mammalian teeth or bones or parts thereof, extracted with an alcoholic hydroxide and reacting positively with an anti-RGD antibody. The invention further provides a bone graft or bone replacement comprising acellular powder of crushed mammalian teeth or bones or parts thereof, extracted with an alcoholic hydroxide, heated to a temperature between 110 and 190° C., and preferably reacting positively with an anti-RGD antibody. In one embodiment, the bone graft or bone replacement of the invention is used as an autograft or an allograft. In an important embodiment, the bone graft or bone replacement of the invention is safely used as a xenograft.

The invention relates to a method of manufacturing a bone graft or bone replacement, comprising steps i) providing a mammalian tooth or bone or a part thereof; ii) immersing said mammalian tooth or bone or a part thereof in alcoholic solution of an alkali hydroxide; iii) washing said tooth or bone or a part thereof after immersing in said alcoholic hydroxide with a saline solution; iv) heating and drying said washed tooth or bone or a part thereof at a temperature of between 110 and 190° C.; and v) crushing said tooth or bone or a part thereof, wherein the step of crushing is performed after any one of steps i) to iv); thereby providing an acellular powder of crushed mammalian teeth or bones or parts thereof for use as a safe bone draft. In a preferred embodiment of the invention, said method comprises i) providing a mammalian tooth or bone or a part thereof, washing and crushing said tooth or bone or a part thereof, ii) immersing said crushed mammalian tooth or bone or a part thereof in an alcoholic solution of an alkali hydroxide for a period of from 0.25 to 5 hour; iii) washing said crushed material after said immersing of step ii) with a saline solution; and iv) heating said crushed and washed material of step iii) at a temperature of between 110 and 190° C. for a period of from 0.1 to 3 hour.

In a preferred embodiment of the invention, said part of the tooth comprises dentine. In one preferred embodiment of the invention, the mammalian teeth are processed to separate fractions containing dentine for further steps. The method of the invention may comprise a step of removing soft tissue before further processing, or removing enamel before further processing, or removing both and performing steps ii) to iv) without these components. In a preferred embodiment of the invention, the process further comprises y-irradiation. Said alcohol comprises lower alcohol, preferably ethanol. Said alkali hydroxide may comprise sodium hydroxide.

Said alcohol is preferably aqueous ethanol, 50-90 vol. %, such as about 70% vol., and said hydroxide is sodium hydroxide in a concentration of up to saturation in said aqueous ethanol at ambient temperature. Said step of immersing is performed preferably between 0.3 and 3 hour, such as between 0.5 and 1 hour, and said step of heating for from 0.2 to 2 hours. Said step of washing may comprise a saline solution, for example 0.15 M NaCl, possibly buffered. The washing may take from 0.75 to 1.5 hours, such as 1 hour. Said step of heating may take between 0.3 and 1 hours at 150° C., such as about 0.5 hours.

The invention aims at providing a safe bone graft or bone replacement, being essentially an acellular powder of crushed mammalian teeth or bones or parts thereof, extracted with an alcoholic hydroxide and heated at a temperature higher than 105° C. and lower than 200° C., preferably between 110 and 190° C., for example between 120 and 180° C., for use in replacing a missing bone in dental surgery, in treating fractures, or after resection of bone tumors.

In an important embodiment, the invention provides the use of a material comprising mammalian teeth or bones or parts thereof treated with an alcoholic hydroxide and heated to a temperature between 110 and 190° C. in the preparation of a bone allograft or xenograft.

### DETAILED DESCRIPTION OF THE INVENTION

It was shown that bone morphogenesis is induced by special peptides, found, for example, in demineralized bone or teeth (see, for example, U.S. Pat. No. 6,677,306). Accordingly, many works attempted to provide bone grafts derived from demineralized bones or teeth, or from materials treated with hydrochloric acid (see already cited EP 2 462 899 and U.S. Pat. No. 9,610,383). It has now been found that a powder obtained from mammalian teeth or parts thereof, comprising dentine and rid of cells, can advantageously serve as a safe bone graft if said teeth or parts thereof are treated with alcoholic hydroxide and heated to a temperature of 110° C. or more, such as 150° C.; it may seem surprising, as the instant conditions do not demineralize the teeth and, moreover, the conditions would supposedly destroy and/or remove the special growth peptides. The inventors believe, without wishing to be limited by any particular theory, that the instant teeth-derived material comprises residual dentine, which supports the bone regeneration. Particularly, the inventors believe that the teeth-derived material contains hydroxyapatite and residual collagen, which collagen, even if being modified by alcoholic hydroxide and by thermal treatment, exhibits osteoinductive effects and together with the hydroxyapatite also osteoconductive effects, the effects resulting from combined structural properties of the modified collagen protein and the inorganic matrix. In one embodiment of the invention, the teeth-derived material is particularly active when providing a positive reaction with antibodies to the Arg-Gly-Asp (RGD) sequence; the RGD sequence is known to be comprised in receptors for cell adhesion molecules, as well as in collagen I which is a part of dentine. In another aspect of the invention, the method of the invention is employed with mammalian bones or parts thereof, providing a superior bone graft or a bone replacement.

Based on the above findings, the present invention is directed to a composition for treating bone fractures, or for replacing a missing bone in dental surgery, or for providing bone after resection of bone tumors. The composition has no toxicity, low immunogenicity, and is free of infectious agents. Particularly, the composition is absolutely free of any infectious or toxic agents, including eukaryotic microorganisms, spores, bacteria, viruses, proteins, lipopolysaccharides, or nucleic acids.

The composition of the invention comprises teeth-derived dentine or bone-derived base treated with alcoholic hydroxide and heated to a temperature of from 110 to 190° C., for example at about 150° C. In one embodiment, the teeth-derived or bone-derived material is heated at two steps at two different temperatures. In a preferred embodiment, the invention provides a composition to be used as a bone graft, derived from mammalian teeth or bones or their parts, for example from teeth rid of enamel and cellular components, exposed to an alkali hydroxide, such as sodium or potassium hydroxide in alcoholic solution, the solution comprising a lower alcohol, optionally mixed with another organic solvent or water. Said alcohol comprises, for example, ethanol. In one embodiment, the composition of the invention positively reacts with anti-RGD antibodies.

The invention will be further described and illustrated in the following examples.

### EXAMPLE

All steps were done in clean hood until blisters packaging stage. The process included grinding the teeth, for example pig teeth, and screening on 300 μm, and re-grinding the greater particles. The teeth were immersed for 40 minutes in sodium hydroxide dissolved in 70 vol % ethanol, 70 wt % NaOH+30 wt % ethanol (70 vol %), while occasionally stirring to release non-dentin material and let it float and removing said floating material with a peristaltic pump. The alcoholic hydroxide was removed, and the crushed teeth were washed with distilled water ten times and allowed to be immersed in PBS for five minutes. The PBS was removed, and the immersion was repeated until the pH of the wash liquid was between 7.5 and 8.5. The crushed and washed teeth were placed on a stainless steel tray, a layer not higher than 1 cm, and heated in an oven for 10 minutes at 180° C. and for 90 minutes at 130° C. If the loss on drying was higher than 5%, the heating at 130° C. was prolonged by 30 minutes.

Desired portions were packed into glass or plastic vials or other medical class materials. If desired, portions were transferred to gamma radiation. No microbial growth was observed in the final package. Bio compatibility testing and the cytotoxicity in vitro tests were performed, and the product complied with the requirements.

While this invention has been described in terms of some specific examples, many modifications and variations are possible. It is therefore understood that within the scope of the appended claims, the invention may be realized otherwise than as specifically described.

1. A bone graft or bone replacement comprising acellular powder of crushed mammalian teeth or bones or parts thereof, extracted with an alcoholic hydroxide and heated to a temperature between 110 and 190° C.

2. The bone graft or bone replacement of claim 1 reacting positively with an anti-RGD antibody.

3. (canceled)

4. The bone graft or bone replacement of claim 1, for use as a xenograft.

5. A method of manufacturing the bone graft or a bone replacement of claim 1, comprising steps
i) providing a mammalian tooth or bone or a part thereof
ii) immersing said mammalian tooth or bone or a part thereof in alcoholic solution of an alkali hydroxide;
iii) washing said tooth or bone or a part thereof after immersing in said alcoholic hydroxide with a saline solution;
iv) heating and drying said washed tooth or bone or a part thereof at a temperature of between 110 and 190° C.; and
v) crushing said tooth or bone or a part thereof, wherein the step of crushing is performed after any one of steps i) to iv);
thereby providing an acellular powder of crushed mammalian teeth or parts thereof.

6. The method of claim 5, comprising steps
i) providing a mammalian tooth or bone or a part thereof, washing and crushing said tooth or bone or a part thereof;
ii) immersing said crushed mammalian tooth or bone or a part thereof in alcoholic solution of an alkali hydroxide for a period of from 0.25 to 5 hours;
iii) washing said crushed material after said immersing of step ii) with a saline solution; and
iv) heating said crushed and washed material of step iii) at a temperature of between 120 and 180° C. for a period of from 0.1 to 3 hour.

7. The method of claim 5, wherein said part of the tooth comprises dentine.

8. The method of claim 5, further comprising a step of removing soft tissue or enamel after step i) and performing steps ii) to iv) without these components.

9. The method of claim 5, further comprising irradiation.

10. The method of claim 5, wherein said alcohol comprises ethanol.

11. The method of claim 5, wherein said alkali hydroxide comprises sodium hydroxide.

12. The method of claim 5, wherein said alcohol is aqueous ethanol, 50-90 vol. %, and said hydroxide is sodium hydroxide in a concentration of up to saturation in said aqueous ethanol at ambient temperature.

13. The method of claim 5, wherein said immersing is performed for from 0.3 to 3 hours, and said step of heating for from 0.2 to 1 hour at 150°.

14. The bone graft or bone replacement of claim 1 for use in replacing a missing bone in dental surgery, in treating fractures, or after resection of bone tumors.

15. A matrix comprising mammalian teeth or bone or parts thereof treated with an alcoholic hydroxide and heated to a temperature of between 110 and 190° C. for use in the preparation of a bone xenograft.
